(19) United States
(12) Patent Application Publication
(10) Pub. No.: US 2006/0018961 A1
(43) Pub. Date: Jan. 26, 2006

(54) Compression formed preparation and method for manufacturing same

(75) Inventors: Naoko Nakamura, Komaki-shi (JP); Naoki Kameyama, Nishikasugai-gun (JP); Akihito Iwasa, Takayama-shi (JP); Yukinari Iwata, Kakamigahara-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Taiyo Yakuhin Co., Ltd., Nagoya-shi (JP)

(21) Appl. No.: 11/236,640

(22) Filed: Sep. 28, 2005

(30) Foreign Application Priority Data
Aug. 1, 2003 (JP) 2003-205474

(51) Int. Cl. A61K 9/20; A61K 31/366

(52) U.S. Cl. 424/464; 514/460

(57) ABSTRACT

A compression formed preparation that rapidly disintegrates in the mouth or in an aqueous solvent, which imparts an excellent feeling during administration, and maintains a suitable hardness required for handling such as distribution and the like, and a method for manufacturing the compression formed preparation. The present invention provides a rapidly disintegrating compression formed preparation comprising one or more compounds selected from gluconolactones and pullulans added to the pharmaceutical base. The pharmaceutical base is preferably a saccharide and particularly preferably a sugar alcohol and starch syrup. The compression formed preparation is prepared by compressing granules obtained by granulation of particles comprising preparation assistants in addition to the pharmaceutical base in a solution of gluconolactone or pullulan dissolved in an aqueous solvent or together with gluconolactone or pullulan in an aqueous solvent.

### BACKGROUND OF THE INVENTION

1. Technical Field

The present invention relates to a compression formed preparation comprising a compression formed composition for pharmaceutical and food use that quickly disintegrates in only a small amount of water and to a method for manufacturing the same.

2. Background Art

Recently, many oral preparations have been provided in the health and nutritional food fields in addition to the pharmaceutical field. Many of these oral preparations are provided in a dry form such as a tablet, capsule, granule, and powder. However, it is hard for elderly people, children, and patients experiencing difficulty in swallowing to take many of these oral preparations.

For these reasons, in order to ease administration of these preparations, dry syrup and the like which form a syrup when suspended in water have been provided. However, when the preparation is in the form of a powder or granule packaged per dose, a proper dose may not be administered. A portion of the preparation may remain in the package or some portion may spill when opening the package.

Recently, in order to solve these problems of difficulty in administration, development of a tablet or lozenge that can be administered without water and can be quickly disintegrated in the mouth and a tablet or lozenge that quickly dissolves in an aqueous solvent have been pursued.

As examples of a method for preparing the tablet or lozenge, a method of forming a tablet rapidly disintegrating in the mouth from a mixture with a water content sufficient for wetting the surface of the particles (for example, Japanese Patent Application Laid-open No. 1993-271054), a method comprising compressing a non-crystalline saccharide as an essential component under low pressure, wetting the obtained tablet under humidification, and further drying the tablet to obtain a tablet rapidly disintegrating in the mouth (for example, Japanese Patent Application Laid-open Nos. 1999-12162 and 1999-349475), and the like can be given. A rapidly disintegrating compressed preparation made from a composition obtained by spray drying a homogenous suspension of a mixture of an inorganic antacid and a saccharide at a ratio of 1:1 by weight is also known (for example, Japanese Patent Application Laid-open No. 2000-86537). A method for preparing a tablet that rapidly disintegrates in the oral cavity comprising a combination of a physiologically active substance, saccharide, and low substituted hydroxypropyl cellulose under fixed conditions (for example, Japanese Patent Application Laid-open No. 2000-103731), a tablet that rapidly disintegrates in the oral cavity comprising a crystallized saccharide and a noncrystallized saccharide and a method for preparing the same (for example, Japanese Patent Application Laid-open No. 2002-154988), and a tablet that rapidly disintegrates in the oral cavity comprising a saccharide coated with a metasilicate aluminate (for example, Japanese Patent Application Laid-open No. 2002-308760) are also known.

Although these conventional preparations rapidly disintegrate in an aqueous solvent and have a hardness sufficient for portability, their manufacturing processes have problems related to the use of water or the requirement for handling the materials in high humidity conditions, which may impair stability of the products depending on the physiologically active substance used. Manufacturing process control is not always satisfactory. Furthermore, preparations using an inorganic antacid have a problem not only of a pH control during the manufacturing process, but also pH change in the digestive tract, because a large amount of the inorganic antacid is used. Moreover, the requirement of a constant pressure for compression in the conventional manufacturing methods makes it difficult to establish proper manufacturing conditions and makes the operation complicated. In addition, since administration of the preparations of the prior art imparts a rough feeling and a powdery feeling in the mouth after the preparation disintegrates, improvement in feeling during use has been desired.

An object of the present invention is to provide a compression formed preparation such as a tablet or lozenge that rapidly disintegrates in the mouth or in an aqueous solvent with ease, provides an excellent feeling during administration, and maintains a hardness sufficient for portability, and to provide a method for preparing the compression formed preparation.

### SUMMARY OF THE INVENTION

As a result of extensive research to achieve the above object, the present inventors have discovered a compression formed preparation such as a tablet or lozenge possessing a strength and hardness sufficient for preventing loss during distribution and rapidly disintegrating in a small amount of water can be obtained by adding one or more types of gluconolactones or pullulans. The inventors have further found that even if prepared using a conventional method, the compression formed preparation possesses a sufficient hardness and rapidly disintegrates in a small amount of water. These findings have led to the completion of the present invention.

Specifically, the present invention provides a rapidly disintegrating compression formed preparation comprising one or more compounds selected from gluconolactones and pullulans.

The above compression formed preparation obtained by adding one or more compounds selected from glucolactones and pullulans to a pharmaceutical base.

The pharmaceutical base in the above compression formed preparation is preferably saccharide.

The content of the compound selected from gluconolactones and pullulans in the above compression formed preparation is preferably from 0.0001 to 0.1 part by mass of the compression formed preparation.

The content of the compound selected from gluconolactones and pullulans in the above compression formed preparation is from 0.0002 to 0.15 part by mass for 1 part by mass of the pharmaceutical base.

The above compression formed preparations preferably further comprises a physiologically active substance.

The above physiologically active substance is preferably a pharmaceutically active component.

The present invention further provides a method for manufacturing a rapidly disintegrating compression formed preparation comprising granulating particles comprising a pharmaceutical base, and if necessary, further comprising other appropriate preparation assistants in a solution comprising one or more compounds selected from gluconolactones and pullulans dissolved in an aqueous solvent or granulating particles comprising a pharmaceutical base, and if necessary, further comprising other appropriate preparation assistants along with one or more compounds selected from gluconolactones and pullulans in an aqueous solvent, and forming the obtained granules into a formed preparation by compression.

Use of one or more compounds selected from gluconolactones and pullulans in the manufacture of a rapidly disintegrating compression formed preparation.

### DETAILED DESCRIPTION OF THE INVENTION AND PREFERRED EMBODIMENTS

The present invention relates to a rapidly disintegrating compression formed preparation which disintegrates in a small amount of water within a short period of time. The compression formed preparation can be obtained by adding one or more compounds selected from gluconolactones and pullulans to a pharmaceutical base, further adding a physiologically active component and other additives such as preparation assistants when necessary to obtain a composition for compression forming, and compressing the composition into the compression formed preparation. The present invention also relates to the method for manufacturing the rapidly disintegrating compression formed preparation.

Based on the position of the lactone ring, the gluconolactone used in the compression formed preparation of the present invention is a γ-gluconolactone or δ-gluconolactone, with δ-gluconolactone being preferable. The pullulan used in the compression formed preparation of the present invention is a natural saccharide comprising α-1,6 bonded maltotriose, which is made from starch.

The gluconolactone is preferably used in an amount of 0.0001-0.1 part by mass, more preferably 0.001-0.07 part by mass, and even more preferably 0.003-0.05 part by mass for 1 part by mass of the compression formed preparation. The gluconolactone is preferably used in an amount of 0.0002-0.15 part by mass, more preferably 0.001-0.07 part by mass, and even more preferably 0.003-0.05 part by mass for 1 part by mass of the pharmaceutical base. The pullulan is preferably used in an amount of 0.0001-0.1 part by mass, more preferably 0.001-0.05 part by mass, and even more preferably 0.003-0.03 part by mass for 1 part by mass of the compression formed preparation. The pullulan is preferably used in an amount of 0.0002-0.15 part by mass, more preferably 0.001-0.07 part by mass, and even more preferably 0.003-0.05 part by mass for 1 part by mass of the pharmaceutical base. When both gluconolactone and pullulan are used together, they are preferably combined at a mass ratio of gluconolactone:pullulan of 0.1-10:0.01-5.

The pharmaceutical base used in the compression formed preparation of the present invention is a base for forming a solid preparation such as a tablet or lozenge. There are no specific limitations to the pharmaceutical base if it comprises an additive known as an excipient of which all or a portion is soluble in water. In view of the objective of the present invention, the pharmaceutical base is particularly preferably a saccharide. A silicic acid compound can also be preferably used as the pharmaceutical base.

Various types of saccharide can be used in the present invention without any specific limitations. As examples of the saccharide, sugar, starch sugar, lactose, honey, sugar alcohol, and the like can be given. Two or more types of these saccharides may be combined in a suitable proportion. As examples of the sugar, white sugar, coupling sugar, fructo-oligosaccharide, palatinose, and the like can be given. As examples of the starch sugar, glucose, maltose, powdered sugar, starch syrup, fructose, and the like can be given. As examples of the lactose, lactose, isomerized lactose (lactulose), reduced lactose (lactitol), and the like can be given. As examples of the honey, various types of commonly edible honey can be given. As examples of the sugar alcohol, sorbitol, mannitol, maltitol, reduced starch sugar compound, xylytol, reduced palatinose, erythritol, and the like can be given. Of these, sugar alcohol and starch syrup are preferable. As the sugar alcohol, mannitol, trehalose, xylytol, and sorbitol are particularly preferable.

As the silicic acid compound used in the present invention, salts of alkali metal or alkaline earth metal of silicic acid or methasilicic acid can be given. Specifically, calcium silicate, magnesium silicate, aluminum silicate, and magnesium aluminometasilicate are preferable.

The rapidly disintegrating compression formed preparation of the present invention may comprise a physiologically active component when necessary. As the physiologically active component, a pharmaceutically active component, flavor component, nutritional component, and the like can be given.

Of these physiologically active components, the pharmaceutically active component is one or more components selected from a nutritional health agent, antipyretic/painkiller/antiphlogistic, psychotropic agent, anti-anxiety agent, antidepressant, sedative hypnotic agent, antispasmodic agent, gastrointestinal drug, antiacid, antitussive expectorant, agent for dental and oral use, antihistamine, anti-allergy agent, cardiotonic, anti-arrhythmic agent, diuretic, antihypertensive, vasoconstrictor, coronary vasodilator, angiotelectasia agent, chloretic drug, antibiotic, chemotherapeutic drug, anti-diabetes agent, anti-osteoporosis drug, myorelaxant, and the like.

As examples of the nutritional health agent, vitamins such as vitamin A, vitamin D, vitamin E (d-α-tocopherol acetic acid and the like), vitamin B₁ (dibenzoyl thiamine, fursultiamine hydrochloride, and the like), vitamin B₂ (riboflavin tetrabutyrate and the like), vitamin B₆ (pyridoxine hydrochloride and the like), vitamin C (ascorbic acid, sodium L-ascorbate, and the like), and vitamin B₁₂ (hydroxocobalamin acetate and the like); minerals such as calcium, magnesium, and iron; proteins; amino acids; oligosaccharides; herbal medicines, and the like can be given.

As examples of the antipyretic/painkiller/antiphlogistic, aspirin, acetaminophen, ethenzamide, ibuprofen, diphenhydramine hydrochloride, dl-chlorpheniramine maleate, dihydrocodeine phosphate, noscapine, methylephedrine hydrochloride, phenylpropanolamine hydrochloride, caffeine, serrapeptase, lysozyme chloride, tolfenamic acid, mefenamic acid, sodium diclofenac, flufenamic acid, salicylamide, aminopyrine, ketoprofen, indomethacin, bucolome, pentazocine, tranexamic acid, and the like can be given.

As examples of the psychotropic agent, chlorpromazine, reserpine, and the like can be given. As examples of the anti-anxiety agent, chlordiazepoxide, diazepam, and the like can be given. As examples of the antidepressant, imipramine, maprotiline, amphetamine, and the like can be given. As examples of the sedative hypnotic agent, estazolam, nitrazepam, diazepam, sodium phenobarbital, triazolam, brotizolam, and the like can be given. As examples of the antispasmodic agent, scopolamine hydrobromide, diphenhydramine hydrochloride, papaverine hydrochloride, and the like can be given.

As examples of the gastrointestinal drug, stomachic digestive agents such as diastase, saccharated pepsin, scopolia extract, lipase AP, and cinnamon oil; antiflatulants such as berberine chloride, lactobacillus and bifidobacteria; and antiemetics such as domperidones can be given. As examples of the antacid, magnesium carbonate, sodium hydrogencarbonate, magnesium aluminometasilicate, synthetic hydrotalcite, precipitated calcium carbonate, magnesium oxide, and the like can be given. Anti-peptic ulcer agents such as gefarnate, cetraxate hydrochloride, teprenone, sofalcone, rebamipide, cimetidine, ranitidine hydrochloride, famotidine, nizatidine, omeprazole, and lansoprazole can also be given. Further, laxatives such as sodium picosulfate, senna extract, and bisacodyl can be given.

As examples of the antitussive expectorant, chloperastine hydrochloride, dextromethorphan hydrobromide, theophyline, potassium guaiacolsulfonate, guaifenesin, and the like can be given. As examples of the agent for dental and oral use, oxytetracycline, triamcinolone acetonide, chlorhexidine hydrochloride, lidocaine, and the like can be given.

As examples of the antihistamine, diphenhydramine hydrochloride, promethazine, isothipendyl hydrochloride, dl-chlorpheniramine maleate, and the like can be given. As examples of the cardiotonic, etilefrine hydrochloride and the like can be given. As examples of the anti-allergy agent, oxamide, tranilast, mequitazine, ketotifen fumarate, epinastine hydrochloride, cetirizine hydrochloride, and the like can be given. As examples of the anti-arrhythmic agent, procainamide hydrochloride, propanolol hydrochloride, pindolol, and the like can be given. As examples of the diuretic, isosorbide, furosemide, and the like can be given. As examples of the antihypertensive, delapril hydrochloride, captopril, hexamethonium bromide, hydralazine hydrochloride, labetalol hydrochloride, methyldopa, and the like can be given.

As examples of the vasoconstrictor, phenylephrine hydrochloride and the like can be given. As examples of the coronary vasodilator, carbocromen hydrochloride, molsidomine, verapamil hydrochloride, and the like can be given. As examples of the angiotelectasia agent, cinnarizine and the like can be given. As examples of the choleretic drug, dehydrocholic acid, torepibutone, and the like can be given.

As examples of the antibiotic, cephem antibiotics such as cefalexin, amoxicillin, pivmecillinam hydrochloride, and cefotiam dihydrochloride, penem antibiotics, carbapenem antibiotics, and the like can be given. As examples of the chemotherapeutic drug, sulfamethizole, thiazolsulfone, and the like can be given. As examples of the anti-diabetes agent, tolbutamide, voglibose, and the like can be given. As examples of an anti-osteoporosis agent, ipriflavone and the like can be given. As examples of the myorelaxant, methocarbamol and the like can be given.

As preferable examples of the physiologically active component to be used in the compression formed preparation of the present invention, vitamins, herbal medicine, antipyretic/painkiller/antiphlogistic, anti-anxiety agent, sedative hypnotic agent, antispasmodic agent, gastrointestinal drug, anti-peptic ulcer agent, antitussive expectorant, antihistamine, anti-allergy agent, antihypertensive, anti-diabetes agent, anti-osteoporosis agent, myorelaxant, and the like can be given. These active components may be diluted with a diluent commonly used in the pharmaceutical and food fields. At least one of the physiologically active components used in the compression formed preparation of the present invention may be in the form of an oil.

There are no limitations to the form or use of the compression formed preparation of the present invention obtained in the above manner. The compression formed preparation may be used in the form of a lozenge or tablet or the like in the pharmaceutical and food fields. However, the compression formed preparation is preferably used as a compression formed preparation that rapidly disintegrates in water, particularly, a small amount of water. Specifically, the compression formed preparation of the present invention disintegrates in a short period of time of usually 1-60 seconds and preferably 1-30 seconds when caused to come in contact with a small amount of water.

The compression formed product of the present invention is prepared by first preparing granules for compression forming. Although the granules for compression forming can be obtained by merely mixing a pharmaceutical base, physiologically active component, and other desirable components, these granules are preferably obtained by wet granulation. In the wet granulation, preferably, a mixture of a pharmaceutical base and, optionally, one or more physiologically active components and other suitable preparation assistants is granulated using a solution of one or more compounds selected from gluconolactones and pullulans dissolved in an aqueous solvent, or a mixture further containing one or more compounds selected from gluconolactones and pullulans is granulated in an aqueous solvent. Alternatively, suitable preparation assistants may be mixed with an aqueous solution of one or more compounds selected from gluconolactones and pullulans. It is also possible to prepare granules comprising the pharmaceutical base and one or more of gluconolactones and pullulans, add a physiologically active component to the granules, and form the resulting granules by compression.

As the aqueous solvent in the wet granulation, an organic solvent exhibiting water miscibility may be used according to the type of physiologically active component and other preparation assistants. As the water miscible organic solvent, ethanol, propanol, and the like are preferable. There are no specific limitations to the granulation method as long as it is a commonly used method. Examples of the granulation method include stirring granulation, fluid bed granulation, rolling granulation, and the like, with fluid bed granulation being particularly preferable.

The obtained granules may be formed by compression into the compression formed preparation of the present invention using common methods such as tabletting. Although the pressure used in the compression forming can be suitably selected in order to obtain a tablet that disintegrates in a small amount of water within 1-60 seconds, from the viewpoint of wear during distribution, a pressure of 1,000-20,000 N is preferably used, with 3,000-15,000 N being particularly preferable. Although the compression formed preparation of the present invention can be produced by a common method such as tabletting or the like, the resulting formed preparation possesses a suitable strength and hardness and does not disintegrate during distribution and storage.

Furthermore, as long as the effect of the present invention is not hindered, various commonly used additives referred to as “other preparation assistants” in the above may be used in the manufacturing of the compression formed preparation of the present invention.

As examples of the preparation assistants, a disintegrating agent, binder, lubricant, filler, foaming agent, sweetener, masking agent, flavoring component, perfume, adjuvant, and the like can be given.

As examples of the disintegrating agent, starches such as corn starch and potato starch, partial alpha starch, sodium carboxymethyl starch, carmellose, carmellose calcium, cross carmellose sodium, polyvinyl alcohol, crospovidone, low substituted hydroxypropyl cellulose, crystalline cellulose, hydroxypropyl starch, and the like can be given. As examples of the binder, hydroxypropyl cellulose, hydroxypropyl methyl cellulose, carboxyvinyl polymer, carmellose sodium, alpha starch, polyvinyl pyrrolidone, gum Arabic, gelatin, pullulan, and the like can be given. As examples of the filler, sucrose, glucose, lactose, mannitol, maltose, sorbitol, calcium phosphate, calcium sulfate, and the like can be given.

As examples of the flavoring component, citric acid, tartaric acid, malic acid, and the like can be given. As examples of the foaming agent, sodium bicarbonate and the like can be given. As examples of the sweetener, sodium saccharin, dipotassium glycyrrhizin, aspartame, stevia, thaumatin, and the like can be given. As examples of the masking agent, water insoluble polymers such as ethyl cellulose, polymers insoluble in saliva and soluble in gastric fluid such as a copolymer of methyl methacrylate, butyl methacrylate, and diethylaminoethyl methacrylate, and the like can be given.

As examples of the perfume, lemon, lemon-lime, orange, menthol, peppermint oil, vanillin, or powders of these absorbed with dextrin or cyclodextrin, and the like can be given. As examples of the lubricant, magnesium stearate, sucrose fatty acid ester, polyethylene glycol, talc, stearic acid, and the like can be given.

As examples of the adjuvant, a coloring agent, physiologically active component stabilizer, solubilizer, and the like can be given. As examples of the coloring agent, food dyes such as food yellow No. 5, food red No. 3, and food blue No. 2, food lake dye, red iron oxide, and the like can be given. As examples of the stabilizer or solubilizer, antioxidants such as ascorbic acid and tocopherol, surfactants such as polysorbate 80, and the like can be given depending on the physiologically active component used.

### EXAMPLES

The present invention will be described in more detail by way of Examples and Test Examples which should not be construed as limiting the present invention.

### Preparation Example

### Preparation of Base Granules

240 g of magnesium aluminometasilicate (trade name: Neusilin FL2, manufactured by Fuji Chemical Industry Co., Ltd.) was suspended in 2,400 g of purified water. Granules of pharmaceutical base were prepared from 2,400 g of the above Neusilin FL2 suspension for 5,484 g of D-mannitol (trade name: Mannit P, manufactured by Towa Chemical Industry Co., Ltd.) in a fluid bed granulating machine (FLO-5 fluid bed granulating machine, manufactured by the Freund Corporation).

### Example 1

### Preparation of Compression Formed Preparation Comprising Gluconolactone

15.32 g of gluconolactone (trade name: Glucono δ lactone, manufactured by Astellas Pharma Inc.) was dissolved in 306.4 g of purified water. 800 g of the pharmaceutical base granules obtained in the Preparation Example were added to this gluconolactone aqueous solution and the mixture was granulated using a fluid bed granulation machine. 2.92 g of crosslinkable polyvinyl pyrrolidone (trade name: Kollidon CL and Crospovidone, manufactured by BASF) and 0.29 g of magnesium stearate (manufactured by Nippon Oil and Fats Co., Ltd.) were added to 46.79 g of the obtained granules. Tablets with a diameter of 8.0 mm were prepared from these granules using an oil press at a pressure of 10,000 N.

### Example 2

### Preparation of Compression Formed Preparation Comprising Pullulan

Tablets with a diameter of 8.0 mm were prepared in the same manner as in Example 1 except for using a pressure of 2,500 N and 15.32 g of pullulan powder (trade name: Pullulan PI-21, manufactured by the Hayashibara Group) instead of 15.32 g of gluconolactone.

### Example 3

### Preparation of Compression Formed Preparation Comprising Gluconolactone and Pullulan

7.66 g of gluconolactone and 7.66 g of pullulan were dissolved in 306.4 g of purified water. 800 g of the pharmaceutical base granules obtained in the Preparation Example were added to the gluconolactone-pullulan aqueous solution and the mixture was granulated using a fluid bed granulation machine. 2.92 g of Kollidon CL and 0.29 g of magnesium stearate were added to 46.79 g of the obtained granules. Tablets with a diameter of 8.0 mm were prepared from these granules using an oil press at a pressure of 15, 000 N.

### Comparative Example 1

Tablets with a diameter of 8.0 mm were prepared in the same manner as in Example 1 except for using 15.32 g of propylene glycol alginate (trade name: Kimiroid, manufactured by Kimica Corporation) instead of 15.32 g of gluconolactone.

### Comparative Example 2

Tablets with a diameter of 8.0 mm were prepared in the same manner as in Example 1 except for using 15.32 g of polyoxyethylene polyoxypropylene glycol (trade name: Adekapruronic F68, manufactured by Asahi Denka Co., Ltd.) instead of 15.32 g of gluconolactone.

### Comparative Example 3

Tablets with a diameter of 8.0 mm were prepared in the same manner as in Example 1 except for using 15.32 g of dextrin (trade name: Dextrin, manufactured by Matsutani Chemical Industry Co., Ltd.) instead of 15.32 g of gluconolactone.

### Comparative Example 4

Tablets with a diameter of 8.0 mm were prepared in the same manner as in Example 1 except for using 15.32 g of starch syrup (trade name: Starch Syrup, manufactured by Nihon Shokuhin Kako Co., Ltd.) instead of 15.32 g of gluconolactone.

### Test Example

Disintegration of each of the tablets prepared in Examples 1-3 and Comparative Examples 1-4 was evaluated by three panelists. Specifically, each of the panelists placed the sample tablets in their mouth and evaluated the disintegration time and conditions. The average of the evaluation results for each panelist is shown in Table 1.

TABLE 1

| | Disintegration | |
|---|---|---|
| Sample | time (seconds) | Condition inside the mouth |
| Example 1 | 15 | Disintegrated with extreme smoothness |
| Example 2 | 23 | Disintegrated with extreme smoothness |
| Example 3 | 12 | Disintegrated with extreme smoothness |
| Comparative | >60 | Tablet too hard to disintegrate |
| Example 1 | | |
| Comparative | 43 | Disintegrated smoothly, however, the core |
| Example 2 | | remained |
| Comparative | 15 | Disintegrated smoothly, however, inside |
| Example 3 | | of mouth remained powdery |
| Comparative | 14 | Disintegrated smoothly, however, the core |
| Example 4 | remained | |

### Example 4

Granules were prepared by a common method using an aqueous solution of 100 g of famotidine as a pharmaceutically active component, 45 g of lactose (trade name: Lactose DMV200M, manufactured by DMV International), and 10.5 g of hydroxypropyl cellulose (trade name: HPC-ssL, manufactured by Nippon Soda Co., Ltd.) dissolved in two liters of purified water. Using the obtained granules and a solution of 46.4 g of a ethyl acrylate-methyl methacrylate copolymer emulsion (trade name: Eudragit NE30D, manufactured by Rohm Co., Ltd.) and 8.4 g of talc (trade name: Talcan Hayashi, manufactured by Hayashi Kasei Co., Ltd.) suspended in 104 g of purified water, granules containing famotidine were prepared using a common method. Using the method of Example 1, tablets were prepared from 6.089 g of the granules containing famotidine and 40.7 g of the granules obtained in Example 1.

### Example 5

Tablets were prepared in the same manner as in Example 4 except for using 100 g of lansoprazole instead of 100 g of famotidine and methacrylate copolymer LD (trade name: Eudragit L30D-55, manufactured by Rohm Co., Ltd.) instead of ethyl acrylate-methyl methacrylate copolymer emulsion.

### Example 6

Tablets were prepared in the same manner as in Example 4 except for using 100 g of triazolam instead of 100 g of famotidine.

### Example 7

Tablets were prepared in the same manner as in Example 4 except that starch sugar was added when preparing of the granules according to Example 1.

### Example 8

### Preparation of Compression Formed Preparation Comprising Gluconolactone

15.32 g of gluconolactone (trade name: Glucono δ lactone, manufactured by Astellas Pharma Inc.) was added to 800 g of the pharmaceutical base granules obtained in the Preparation Example. 50.9 g of crosslinkable polyvinyl pyrrolidone (trade name: Kollidon CL and Crospovidone, manufactured by BASF) and 5.1 g of magnesium stearate (manufactured by Nippon Oil and Fats Co., Ltd.) were mixed using a common method. Using the obtained granules, tablets with a diameter of 8.0 mm were prepared using an oil press at a pressure of 10,000 N.

All of the tablets obtained in Examples 4-8 disintegrated in the mouth within 60 seconds and provided a smooth feeling.

The compression formed preparation of the present invention rapidly dissolves in a small amount of water, for example, within 60 seconds when placed in the mouth, and does not impart an unpleasant feeling such as roughness after disintegrating. Further, since the formed preparation possesses a suitable strength and hardness, the preparation has a only minimal risk of disintegration during distribution and storage.

What is claimed is:

1. A rapidly disintegrating compression formed preparation comprising one or more compounds selected from gluconolactones and pullulans.

2. The rapidly disintegrating compression formed preparation according to claim 1, obtained by adding one or more compounds selected from gluconolactones and pullulans to a pharmaceutical base.

3. The rapidly disintegrating compression formed preparation according to claim 2, wherein the pharmaceutical base is a saccharide.

4. The rapidly disintegrating compression formed preparation according to claim 1 or claim 2, wherein the compound selected from gluconolactones and pullulans is contained in an amount from 0.0001 to 0.1 part by mass of the compression formed preparation.

5. The rapidly disintegrating compression formed preparation according to claim 2, wherein the compound selected from gluconolactones and pullulans is contained in an amount from 0.0002 to 0.15 part by mass for 1 part by mass of the pharmaceutical base.

6. The rapidly disintegrating compression formed preparation according to any one of claim 1 or claim 2, further comprising a physiologically active substance.

7. The rapidly disintegrating compression formed preparation according to claim 6, wherein the physiologically active substance is a pharmaceutically active component.

8. A method for manufacturing a rapidly disintegrating compression formed preparation comprising granulating particles comprising a pharmaceutical base and, optionally, other preparation assistants in a solution comprising one or more compounds selected from gluconolactones and pullulans dissolved in an aqueous solvent or granulating particles comprising a pharmaceutical base and, optionally, other preparation assistants along with one or more compounds selected from gluconolactones and pullulans in an aqueous solvent, and forming the obtained granules into a formed preparation by compression.
